(19) United States
(12) Patent Application Publication
(10) Pub. No.: US 2024/0038387 A1
(43) Pub. Date: Feb. 1, 2024

(54) PHYSIOLOGICAL CONDITION INFORMATION FOR REMOTE HEALTHCARE DETERMINATION

(71) Applicant: POPS! DIABETES CARE, INC., Stillwater, MN (US)

(72) Inventors: Lonny Stormo, STILLWATER, MN (US); Daniel William Davis, HUGO, MN (US); Curtis Jerome Christensen, STILLWATER, MN (US); Jennifer E. Englund, MINNEAPOLIS, MN (US); Timothy Balder, STILLWATER, MN (US)

(21) Appl. No.: 18/483,632

(22) Filed: Oct. 10, 2023

Related U.S. Application Data
(63) Continuation of application No. 18/179,529, filed on Mar. 7, 2023.
Application No. 18/179,529 is a continuation of application No. 17/152,433, filed on Jan. 19, 2021, now abandoned.
Application No. 17/152,433 is a continuation of application No. 16/156,446, filed on Oct. 10, 2018, now abandoned.
(60) Provisional application No. 62/570,299, filed on Oct. 10, 2017.

(51) Int. Cl. G16H 40/67; G06Q 10/1093; G16H 20/10; G16H 10/60; G16H 80/00

(52) U.S. Cl. CPC G16H 40/67; G06Q 10/1095; G16H 20/10; G16H 10/60; G16H 80/00; G06Q 40/08

(57) ABSTRACT

Devices, systems, and processes are disclosed herein relating to the collection, transmission, and/or use of physiological condition information (e.g., blood glucose information) for a remote healthcare determination. One embodiment includes a physiological condition information system. This system embodiment includes a physiological condition management device and a remote server. The physiological condition management device can be at a first location (e.g., accompanying a patient at the first location) and the remote server can be at a second location that is different from the first location. The physiological condition management device and remote server can be in data communication over a transmission link. The physiological condition management device is configured to collect physiological condition information associated with the patient and send this information over the transmission link to the remote server. Upon receiving the physiological condition information, the remote server is configured to send a data communication to a remote healthcare provider.

### PRIORITY CLAIM

This application is a continuation of U.S. patent application Ser. No. 18/179,529 filed Mar. 7, 2023, which is a continuation of U.S. patent application Ser. No. 17/152,433 filed Jan. 19, 2021, which is a continuation of U.S. patent application Ser. No. 16/156,446 filed Oct. 10, 2018, which claims priority to U.S. Provisional Patent Application No. 62/570,299 filed Oct. 10, 2017, the entire contents of each application is hereby incorporated by reference in their entirety.

### TECHNICAL FIELD

This disclosure generally relates to the collection, transmission, and/or use of physiological condition information for a remote healthcare determination.

### BACKGROUND

An individual with any one of a number of certain medical conditions may be required to make periodic visits in-person to a healthcare provider. This can be true regardless of whether the current state of the individual's medical condition actually needs medical attention. As one example, various licensing agencies require a healthcare provider to sign off on an authorization indicating that the individual has demonstrated sufficient and stable control of a medical condition in order for a license to be issued to the individual. These in-person visits to a healthcare provider can be burdensome to the individual but yet routinely necessary if the individual wants to obtain, and maintain, the license.

### SUMMARY

In general, various exemplary embodiments relating to the collection, transmission, and/or use of physiological condition information (e.g., blood glucose information) for a remote healthcare determination are disclosed herein. These embodiments may be useful, for instance, in collecting patient physiological condition information that can be used by a remote healthcare provider (e.g., a remote diabetes care provider) in making a remote healthcare provider determination. This remote healthcare provider determination can be made without requiring the patient to be present in-person at the healthcare provider. Moreover, patient physiological condition information may be able to be collected frequently at preset intervals so that the healthcare provider has a more complete picture of the patient's health condition when rendering the healthcare determination as compared to patient information acquired only during in-person visits.

For example, a remote healthcare provider can receive a request for a healthcare provider authorization for a particular patient who is not present at the healthcare provider. Physiological condition information associated with this patient can be sent to the remote healthcare provider and used by the remote healthcare provider in making the requested healthcare provider authorization. One example includes a healthcare provider authorization that the patient has demonstrated sufficient and stable control of the patient's medical condition. This authorization may then be received by a remote healthcare determination receiver, such as a licensing body, and used in making a determination (e.g., granting a license) that depends on a medical condition of the individual seeking the license.

In another example, physiological condition information associated with a patient can be collected and used in remotely assigning a medical professional. For instance, the collected physiological condition information associated with a patient can be processed to select a particular level of medical profession specialization suited for the patient in a future in-person visit. For instance, the processed physiological condition information may indicate that the current state of the patient's medical condition is not at a level requiring medical attention from a highly specialized medical professional during a future in-person visit.

One exemplary embodiment includes a physiological condition management device. The physiological condition management device includes a monitoring device that is configured to collect physiological condition information associated with a patient. The physiological condition management device further includes a non-transitory computer-readable storage article having computer-executable instructions stored thereon to cause at least one programmable processor thereof to receive collected physiological condition information associated with the patient from the monitoring device and transmit this information to a remote server (e.g., a diabetes care system). In a further embodiment, the collected physiological condition information associated with the patient is sent to the remote server along with a request for a healthcare provider authorization. In an additional embodiment, the computer-executable instructions stored thereon cause at least one programmable processor to receive, from the remote server, an action taken by a remote healthcare provider in response to the requested healthcare provider authorization.

Another exemplary embodiment includes a physiological condition information system. This system embodiment includes a physiological condition management device and a remote server. The physiological condition management device can be at a first location (e.g., accompanying a patient at the first location) and the remote server can be at a second location that is different from the first location. The physiological condition management device and remote server can be in data communication over a transmission link. The physiological condition management device is configured to collect physiological condition information associated with the patient and send this information over the transmission link to the remote server. Upon receiving the physiological condition information, the remote server is configured to identify a patient profile corresponding to the received physiological condition information. Based on the patient profile, the remote server is configured to send a data communication to a remote healthcare provider. This sent data can include, for instance, physiological condition information received from the physiological condition management device along with a request for a healthcare provider authorization. In a further embodiment, the remote server can be configured to receive an action taken by a remote healthcare provider in response to the requested healthcare provider authorization. Upon receiving the action taken by the remote healthcare provider, the remote server may also be configured to use the patient profile to send a data communication including information related to the action taken by the remote healthcare provider to a remote healthcare determination receiver and/or the physiological condition management device.

Another exemplary embodiment includes a process for acting on a prompt for a healthcare provider authorization. In this embodiment, the process includes receiving a prompt for a healthcare provider authorization concerning a particular remotely located patient. The process also includes receiving physiological condition information associated with the remotely located patient. In one case, the prompt for the healthcare provider authorization can be received along with the physiological condition information associated with the remotely located patient. The process further includes acting on the prompt for a healthcare provider authorization concerning the remotely located patient. Acting on the prompt may include granting the requested healthcare provider authorization if so warranted by the received physiological condition information associated with the remotely located patient.

The details of one or more examples are set forth in the accompanying drawings and the description below. Other features, objects, and advantages will be apparent from the description and drawings.

### BRIEF DESCRIPTION OF DRAWINGS

The following drawings are illustrative of particular embodiments of the present invention and therefore do not limit the scope of the invention. The drawings are intended for use in conjunction with the explanations in the following description. Embodiments of the invention will hereinafter be described in conjunction with the appended drawings, wherein like numerals denote like elements.

FIG. 1 is a schematic diagram of an illustrative virtual clinic.

FIG. 2 is a schematic diagram of an exemplary embodiment of a physiological condition information system.

FIG. 3 is a schematic diagram of an exemplary embodiment of a blood glucose management system.

FIG. 4 is a flow diagram of an exemplary embodiment of a process of making a request of a remote diabetes care provider.

FIG. 5 a schematic diagram of a decision tree for responding to requests based on physiological and/or other information.

FIG. 6 is a flow diagram of an exemplary embodiment of a process for acting on a prompt for a healthcare provider authorization.

FIG. 7 is a flow diagram of an exemplary embodiment of a process for automatically assigning a medical professional to a patient.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Those skilled in the art will recognize that many of the noted examples have a variety of suitable alternatives.

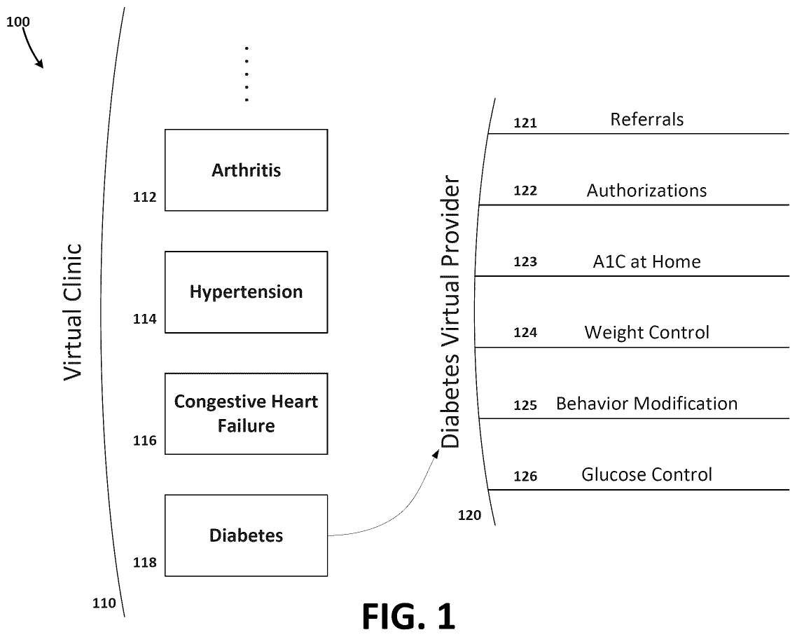

FIG. 1 illustrates a virtual clinic system 100 through which a user may receive several forms of care from several kinds of care providers without having to meet with any of the care providers in person. The left side of FIG. 1 shows the virtual clinic level 110 of the virtual clinic system 100, and the right side of FIG. 1 drills down one level to that of the diabetes virtual provider 120.

In some embodiments, the virtual clinic 110 can include one or more kinds of virtual care providers. For example, the virtual clinic 110 can include an arthritis virtual provider 112, a hypertension virtual provider 114, a congestive heart failure virtual provider 116, a diabetes virtual provider 118, and/or any other care provider that can provide care without requiring a user/patient to meet with the care provider in person. Such virtual care providers can include a physician, a nurse, a non-medical employee (e.g., a scheduler), or other individuals who provide care to users/patients. In some embodiments, an algorithm that receives inputs and provides care to users/patients can be a virtual care provider.

The diabetes virtual provider 120 can provide one or more kinds of care to a user/patient without requiring the user/patient to meet with a diabetes care provider in person. In many embodiments, the diabetes virtual provider 120 can provide care to users/patients who have diabetes or pre-diabetes. In some embodiments, the diabetes virtual provider 120 can provide a referral 121 to a user/patient without requiring an in-person meeting. If a user/patient wants to see an endocrinologist or other diabetes specialist, he/she often needs a referral from a primary care provider. In many embodiments, the diabetes virtual provider 120 can analyze relevant information related to the user/patient and make a referral 121 (or decline to make a referral) without requiring the user to visit a primary care provider in person.

In some embodiments, the diabetes virtual provider 120 can provide an authorization 122 to a user/patient without requiring an in-person meeting. In some instances, an authorization 1220 can be confirmation that the user/patient has demonstrated sufficient and stable control of his/her diabetes or pre-diabetes. Users/patients commonly request such authorizations for purposes of renewing a license (e.g., a driver's license). In some instances, an authorization 122 can be a renewal of a prescription. The diabetes virtual provider can analyze information associated with a user/patient and determine whether the prescription should be renewed or not.

Embodiments of the diabetes virtual provider can provide additional kinds of care to users/patients without requiring in-person visits. For example, the diabetes virtual provider 120 can assist a user/patient in taking his/her A1C measurement at home 123, in controlling his/her weight 124, in modifying his/her diabetes-related behavior 125, and/or in controlling his/her glucose levels 126. In many instances, a user who would conventionally be required to visit a diabetes provider for any of these kinds of care may be able to obtain such care through the diabetes virtual provider 120.

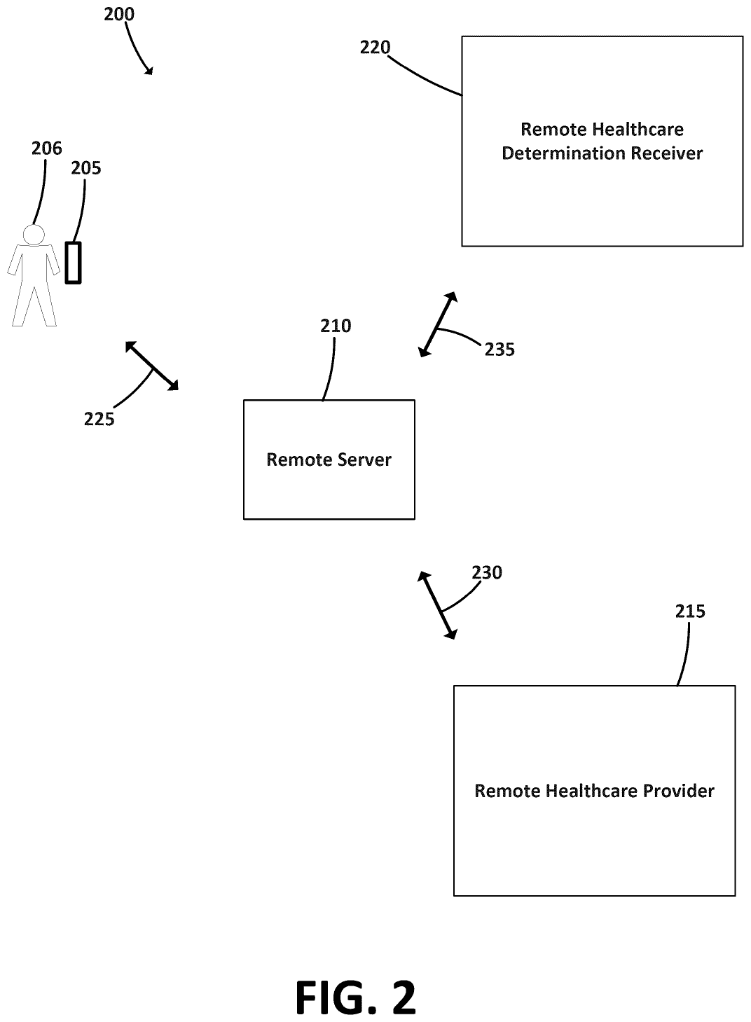

FIG. 2 illustrates a schematic diagram of an exemplary embodiment of a physiological condition information system 200. Components of the system 200 can include a physiological condition management device 205 and a remote server 210. In some further examples, the system 200 can additionally include a remote healthcare provider 215 and/or a remote healthcare determination receiver 220. The use of the term “remote” generally refers to the location of the particular referenced system component as being remote relative to a patient 206. As described further below, in many cases the device 205 locally accompanies the patient 206, and thus, in such cases, the use of the term “remote” would also generally refer to the location of the particular referenced system component as being remote relative to the device 205.

The system 200, including some or all of the described components, can collect and transmit physiological condition information for use in making a remote healthcare determination. For instance, physiological condition information associated with the patient 206 can be collected locally at the patient 206 (e.g., via the device 205), transmitted to one or more remote system components, and used at one or more remote system components in making a healthcare determination. In certain applications, once the remote healthcare determination has been made, the system 200 can transmit the remote healthcare determination. For instance, where the remote healthcare provider 215 has made the remote healthcare determination, the system 200 may transmit this healthcare determination from the remote healthcare provider 215 to the remote server 210, the device 205, and/or the remote healthcare determination receiver 220. In another instance, where the remote server 210 has made the remote healthcare determination, the system 200 may transmit this healthcare determination from the remote server 210 to the device 205, the remote healthcare provider 215, and/or the remote healthcare determination receiver 220.

As noted, the system 200 can include the physiological condition management device 205. The device 205 can include, for instance, a mobile computing device carried by the patient 206. As examples, the mobile computing device may be a smartphone, laptop, or other personal computing device. The device 205 can include, for instance, a user interface, a network communication mechanism (e.g., one or more wireless transceivers), a processor, and a non-transitory computer-readable storage article. The non-transitory computer-readable storage article can have computer-executable instructions stored thereon and run by the processor to cause the processor to perform specified actions. Such actions can include, for instance, generating prompts on the user interface, collecting physiological condition information associated with the patient 206, transmitting this physiological condition information, and/or receiving a data transmission from a remote system component. In some embodiments, a user may self-report one or more physiological condition characteristics (e.g., behaviors, health conditions, mental conditions, etc.) using the device 205.

In some cases, the device 205 can include a monitoring device. The monitoring device can be configured to collect any physiological condition information associated with the patient 206. As such, when present, the monitoring device can facilitate the collection of physiological condition information associated with the patient 206 by the device 205. In some cases, the device 205 includes multiple, different monitoring devices. The monitoring device(s) can include any component useful in ascertaining a physiological condition of the patient 206. Where the device 205 is a mobile computing device carried by the patient 206, the monitoring device can be integral to the mobile computing device and/or a separate component that is in communication with the mobile computing device. Examples of integral monitoring devices include a camera, an activity measurement mechanism (e.g., an accelerometer), and a location determination mechanism (e.g., GPS component). Moreover, in certain instances, a user interface (e.g., touchscreen, keypad, microphone, etc.) of the device 205 can serve as a monitoring device in that the patient 206 can input physiological condition information associated with the patient at the user interface. This may include the patient 206 self-reporting on one or more physiological condition statuses via such input. Such self-reporting may be in the form of the patient 206 periodically providing physiological condition statuses via input at the device 205 on the patient's own volition or in response to one or more prompts provided at the device 205. Self-reporting on one or more physiological condition statuses via patient input at the device 205 can include, for instance, reporting one or more statuses associated with the patient's behavior, physical condition, and/or mental condition. Examples of separate monitoring devices in communication with the mobile computing device include a heart rate monitor and a blood characteristic analytical device (e.g., a blood glucose management device). Any one or more separate monitoring devices may be in communication with the mobile computing device via a wireless communication channel, such as Bluetooth, radio, or Infrared channels as examples.

In some cases, it may be useful to collect two or more physiological condition information inputs associated with the patient 206. The two or more physiological condition information inputs associated with the patient 206 can be different, for instance, in the sense that the inputs are collected at different times and/or that the inputs relate to different types of physiological condition information. The particular types of physiological condition information inputs can vary depending on the particular patient 206, the particular healthcare provider, the type of remote healthcare provider determination being requested, and/or the particular remote healthcare determination receiver. For example, certain embodiments disclosed herein may provide the capability to run various algorithms for combining two or more different physiological condition information inputs and outputting a determination based on these inputs (e.g., at the device 205, at a remote server, etc.). One particular, illustrative example of this could include an algorithmic combination for calculating one or more results pertaining to the patient's overall healthcare state based on any two or more physiological condition information inputs, including those described herein. In a further particular, illustrative example the algorithmic combination can calculate one or more results pertaining to the patient's subjective motivation for controlling a physiological condition based on any two or more physiological condition information inputs, including those described herein (e.g., based on provided prompts and responsive patient input, in addition to one or more results pertaining to the patient's overall healthcare state). In certain instances, running such algorithm(s) for combining two or more different physiological condition information inputs may include outputting a determination relating to a categorization of the patient in one of a number of different categories each based on the patient's physiological condition information.

Some illustrative examples of a device that may serve as the device 205, or as the monitoring device included with the device 205, are described in U.S. Pat. Nos. 9,380,970; 9,237,866; 8,647,357; US Pat. App. Pub. No. 2017/0231542; US Pat. App. Pub. No. 2017/0143244; US Pat. App. Pub. No. 2016/0328527; US Pat. App. Pub. No. 2016/0324481; US Pat. App. Pub. No. 2016/0324464; US Pat. App. Pub. No. 2016/0302708; US Pat. App. Pub. No. 2016/0120452; and US Pat. App. Pub. No. 2016/0100785. The entire content of each of these publications is hereby incorporated by reference.

As noted, in addition to the device 205, the system 200 can include the remote server 210 (e.g., a diabetes care system). The remote server 210 can be located at a second location, such as a central monitoring station, that is remote from a first location of the device 205 and the patient 206. The remote server 210 can be in communication with the device 205 via a transmission link 225. For instance, the device 205 and remote server 210 can be in two-way communication over the transmission link 225. The transmission link 225 can take any of a variety of suitable forms that allow for data communication between the device 205 and the remote server 210. For instance, the transmission link 225 can take the form of a wireless communication channel, such as a wireless network (e.g., a wireless local area network (e.g., Wi-Fi) or a wireless wide area network (e.g., a cellular communication network)).

The remote server 210 can include one or more components, such as a network communication mechanism (e.g., a wireless transceiver), a processor, and a non-transitory computer-readable storage article. The non-transitory computer-readable storage article of the remote server 210 can have computer-executable instructions stored thereon and run by the processor to cause the processor to perform specified actions. Such actions can include, for instance, generating and sending a prompt (e.g., to the device 205), processing received physiological condition information associated with the patient 206, transmitting physiological condition information (or related information based thereon) and/or receiving a data transmission from a remote system component.

In some embodiments, the remote server 210 can store one or more patient profiles. Each of the one or more patient profiles can be associated with a particular patient, including a patient profile associated with the patient 206. The patient profile can include any information pertaining to the patient 206. Such information can include information identifying the patient 206 (e.g., name, patient number, etc.), healthcare provider information for the patient 206 (e.g., healthcare provider identifying information, listing of specific medical professionals (e.g., including corresponding degrees of specialization of these medical professionals), etc.), physiological condition records of the patient 206 (e.g., past physiological condition information associated with the patient 206), insurance information, and/or healthcare provider authorizations needed by the patient 206 (e.g., a type of healthcare provider authorization needed by the patient 206 and the frequency at which such authorization is needed).

The remote server 210 can use the patient profile associated with the patient 206 to facilitate data communications related to the patient 206 within the system 200. For example, the remote server 210 can be configured to receive from the device 205 physiological condition information associated with the patient 206 and identify a patient corresponding to the received physiological condition information. The received information can be input into the identified patient profile. The remote server 210 can be configured to process the received information, either in isolation or in conjunction with other information held in the identified patient profile. For instance, the received information can be processed in conjunction with other information in the patient profile and used to send a data communication from the remote server 210 to the remote healthcare provider 215. In another example, the remote server 210 can be configured to send a prompt to the device 205 based on information stored in the patient profile associated with the patient 206.

As noted, in certain cases, the system 200 can further include the remote healthcare provider 215 (e.g., a diabetes care provider). In some embodiments, the remote healthcare provider 215 can be one or more specific medical professionals for the patient 206. The remote healthcare provider 215 can be located at a location that is remote from the first location of the device 205 and the patient 206. In some instances, the remote healthcare provider 215 may also be at a location that is remote from the location of the remote server 210. The remote server 210 can be in communication with the remote healthcare provider 215 via a transmission link 230. In some instances, the remote healthcare provider 215 and remote server 210 are in two-way communication over the transmission link 230. The transmission link 230 can take any of a variety of suitable forms that allow for data communication between the remote server 210 and the remote healthcare provider 215. For instance, the transmission link 230 can take the form of a wireless communication channel.

As explained elsewhere herein, various types of data pertaining to the patient 206 can be transmitted between the remote server 210 and the remote healthcare provider 215. In one example, the remote server 210 can use the identified patient profile to send a particular data transmission to the remote healthcare provider 215. This data transmission may include, for instance, physiological condition information associated with the patient 206 (e.g., received from the device 205, including any of the information described herein and, in some cases, records of past physiological condition information received from the device 205) and/or an identification of a particular type of healthcare provider authorization needed by the patient 206 from the remote healthcare provider 215. In such example, this data transmission from the remote server 210 may allow the remote healthcare provider 215 to make the requested healthcare provider authorization based on the included physiological condition information associated with the patient 206 without needing the patient 206 present at the remote healthcare provider 215. This data transmission may include, additionally or alternatively, a request to make an appointment with a specific medical professional at the remote healthcare provider 215.

In various examples, the remote healthcare provider 215 can send one or more data transmissions to the remote server 210, including in some cases to the patient 206 via the remote server 210. One such data transmission from the remote healthcare provider 215 can include the requested healthcare provider authorization. Another example of a data transmission from the remote healthcare provider 215 may include a referral for the patient 206 to another healthcare provider, for instance based on review of the physiological condition information associated with the patient 206 received from the device 205 and/or remote server 210. A further example of a data transmission from the remote healthcare provider 215 can include an approval of one or more algorithms for combining two or more different physiological condition information inputs associated with the patient 206 and outputting a determination based on these inputs. In this example, once the approval from the remote healthcare provider 215 is received, the remote server 210 may be able to provide certain remote healthcare determinations relating to the patient 206 without further input from the remote healthcare provider 215. In some cases, the remote healthcare provider 215 may generate and send a notification to the patient 206 via the remote server 210. A notification generated and sent by the remote healthcare provider 215 to the patient 206 can include, for example, one or more of a prompt for a healthcare provider authorization, a prompt to make an appointment with a specific medical professional, or a prompt to provide additional physiological condition information associated with the patient 206.

In various embodiments, the remote healthcare provider 215 can be one or more specific medical professionals for the patient 206. As one example, the remote healthcare provider 215 can be a single healthcare provider, such as a single clinic or a common healthcare system of providers. As another example, the remote healthcare provider 215 could be both a primary healthcare provider (e.g., a primary or general care clinic) for the patient 206 and one or more specialty healthcare providers (e.g., a specialty clinic, such as an endocrinologist, an ophthalmologist, cardiologist, etc.) for the patient 206. In such an example, the remote server 210 can be in communication with each provider so as to facilitate the exchange of data between the providers, the providers and the device 205, and/or the providers and the remote healthcare determination receiver. Thus, the remote server 210 can be configured to allow for interaction among these system components. For instance, the remote server 210 may be configured to transmit physiological condition information associated with the patient 206 to each of the providers (e.g., including determining which of such information should go to each provider), transmit notifications initiated by any of the providers to the device 205, transmit data between the providers (e.g., a patient referral), and/or transmit a remote healthcare determination from each of the providers to the remote healthcare determination receiver.

As also noted, in some embodiments, the system 200 can additionally include the remote healthcare determination receiver 220. In some embodiments, the remote healthcare determination receiver 220 can be any actor that takes an action related to the patient 206 based on a healthcare provider authorization. In some cases, the remote healthcare determination receiver 220 may also take action using physiological condition information pertaining to the patient 206. This could include the device 205 being in communication with the remote healthcare determination receiver 220 via the remote server 210 so as to receive physiological condition information associated with the patient 206. This may allow for the remote healthcare determination receiver 220 to perform other actions based on the received physiological condition information associated with the patient 206 without needing the patient 206 to be physically present at the remote healthcare determination receiver 220. Such actions may include, for instance, conducting remote consults and/or changing one or more patient medications remotely.

The remote healthcare determination receiver 220 can be located at a location that is remote from the first location of the device 205 and the patient 206. In some instances, the remote healthcare provider 215 may also be at a location that is remote from the location of the remote server 210 and/or the remote healthcare provider 215. The remote server 210 can be in communication with the remote healthcare determination receiver 220 via a transmission link 235. In some instances, the remote healthcare determination receiver 220 and remote server 210 are in two-way communication over the transmission link 235. The transmission link 235 can take any of a variety of suitable forms that allow for data communication between the remote server 210 and the remote healthcare determination receiver 220. For instance, the transmission link 235 can take the form of a wireless communication channel.

As explained elsewhere herein, various types of data pertaining to the patient 206 can be transmitted between the remote server 210 and the remote healthcare determination receiver 220. In one example, the remote server 210 can send to the remote healthcare determination receiver 220 a healthcare provider authorization provided by the remote healthcare provider 215. The remote healthcare determination receiver 220 can use this received healthcare provider authorization to take an action related to the patient 206. Such action can vary depending on the type of healthcare determination receiver 220. For example, where the healthcare determination receiver 220 is a licensing agency such action may include granting or renewing a license. As another example, where the healthcare determination receiver 220 is a pharmacy, such action may include filling a prescription. As an additional example, where the healthcare determination receiver 220 is an employer, such action may include verifying a patient drug test. In certain embodiments, the remote server 210 can send to the remote healthcare determination receiver 220 physiological condition information pertaining to the patient 206 along with the healthcare provider authorization. In some examples, the remote healthcare determination receiver 220 can send to the remote server 210 a data communication related to the action taken by the remote healthcare determination receiver 220.

Accordingly, the system 200 can have a variety of uses. Certain applications of the system 200 may be useful in allowing for a healthcare provider authorization to be made in regards to the patient 206 by an appropriate medical professional without requiring the patient 206 to be physically present at the location of the medical professional. The system 200 can collect and transmit physiological condition information associated with the patient 206 as needed for the medical professional to render the healthcare authorization. Moreover, in certain embodiments, the physiological condition information associated with the patient 206 can be collected by the system 200 (e.g., at the device 205 and/or at the remote server 210) regularly over a prolonged period of time. The frequency and period of time over which the physiological condition information associated with the patient 206 is collected can vary as appropriate for the particular remote healthcare authorization that is to be made. In this way, the system can provide a medical professional with physiological condition information associated with the patient 206 that affords a more complete picture of the patient 206 as compared to infrequent, in-person visits with the patient 206. This may be particularly true where the remote healthcare authorization relates to a medical professional's determination that the patient 206 has demonstrated sufficient and stable control of a medical condition, for instance as required by certain licensing bodies in granting or renewing a license to the patient 206 or pharmacies in filling a prescription for the patient 206.

In certain cases, in addition to the physiological condition information associated with the patient 206, the system can receive and transmit bio-identification information from the patient 206. This may be useful in verifying that the collected physiological condition information is actually that of the patient 206. Such bio-identification information can include any biological information that can be used in authenticating a particular patient's identity. This may include, for example, finger print data, facial recognition data, implanted chip data signal reception, or any other suitable bio-identification information that can be provided by the patient 206. In some embodiments, DNA can be verified from the biological sample used to collect the physiological condition information.

In some applications of the system 200, requests for a healthcare authorization can be made in an automated manner. For example, information can be input into the patient profile, held at the remote server 210, related to one or more particular healthcare authorizations needed by the patient 206. This input information may include the type of healthcare authorization needed and the frequency with which the healthcare authorization is needed. The system 200 can be configured, for instance by executing computer-readable instructions stored at the remote server 210, to use this input to determine what type of physiological condition information to collect from the patient 206 (e.g., via the device 205) and how often the determined physiological condition information should be collected. Using the patient profile, the remote server 210 can transmit a request, at a preset time, for a healthcare authorization to the remote healthcare provider 215 that includes the determined physiological condition information. The remote server 210 may also be configured to receive the healthcare determination from the remote healthcare provider 215 and use the patient profile to convey the healthcare authorization to the remote healthcare determination receiver 220 so that the remote healthcare determination receiver can take an action related to the patient 206 using the healthcare provider authorization. In this way, the patient 206 may be able to have actions taken by the remote healthcare determination receiver 220 that require a healthcare authorization from a medical professional without needing to make an in-person visit to the remote healthcare provider 215 and/or the remote healthcare determination receiver 220.

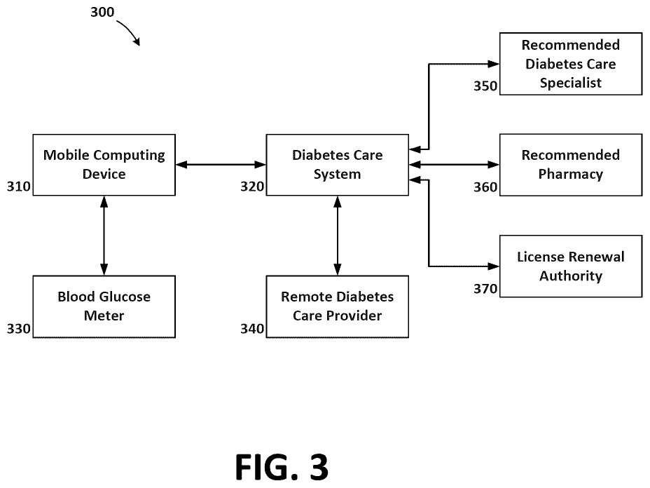

FIG. 3 shows a blood glucose management system 300 that is similar to the system 200 of FIG. 2. Referring to FIG. 3, the system 300 includes a mobile computing device 310 in communication with a diabetes care system 320 via one or more of the communications links described elsewhere herein. A software application may run on the mobile computing device 310. The software application may receive information from a blood glucose meter 330 as described elsewhere herein. The diabetes care system 320 may be a remote server like those discussed elsewhere herein. The diabetes care system 320 may receive information (e.g., blood glucose information, physiological and/or behavioral information, etc.) from the mobile computing device 310 (e.g., through the software application). In some embodiments, the diabetes care system 320 can provide the raw information to a remote diabetes care provider 340. In some embodiments, the diabetes care system 320 can analyze the information (e.g., by running one or more algorithms on the information) and provide the analyzed information to the remote diabetes care provider 340.

The diabetes care system 320 can interact with one or more remote healthcare determination receivers. The diabetes care system 320 can recommend a diabetes care specialist 350 based on input from the remote diabetes care provider 340 and/or on information received from the mobile computing device 310 and/or on information stored at the diabetes care system 320 and/or on other factors. The diabetes care system 320 can interact with the diabetes care specialist 350 as described elsewhere herein. The diabetes care system 320 can recommend a pharmacy 360 based on input from the remote diabetes care provider 340 and/or on information received from the mobile computing device 310 and/or on information stored at the diabetes care system 320 and/or on other factors. The diabetes care system 320 can interact with the recommended pharmacist 360 as described elsewhere herein. The diabetes care system 320 can communicate with a license renewal authority 370 based on input from the remote diabetes care provider 340 and/or on information received from the mobile computing device 310 and/or on information stored at the diabetes care system 320 and/or on other factors. The diabetes care system 320 can interact with the license renewal authority 370 as described elsewhere herein.

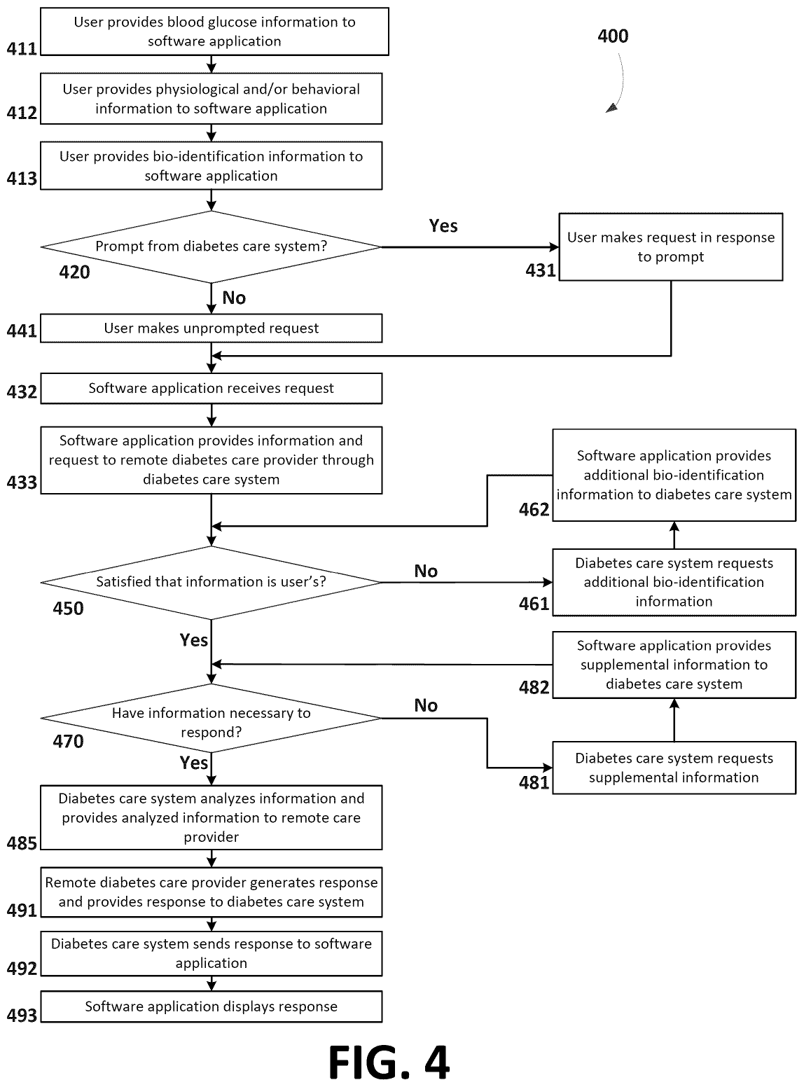

FIG. 4 shows an illustrative method 400 of interacting with a remote diabetes care provider using a software application operating on a mobile computing device. One or more steps in method 400 may be optional in some embodiments. A primary advantage of methods like method 400 is that a user may receive service from a diabetes care provider without having to make an in-person visit to the diabetes care provider, resulting in more efficient care for both the user and the diabetes care provider. In many instances, the software application can allow a user to interact with the remote diabetes care provider without the user having to visit the remote diabetes care provider. The method can include receiving blood glucose information from a user at the software application. In the method, the user can provide the blood glucose information to the software application (411). The blood glucose measurements can be collected from the user over a period of time (e.g., several weeks, months, years, etc.). In some embodiments, the software application can receive the blood glucose information wirelessly from a blood glucose meter. In some embodiments, a blood glucose meter can be connected to the mobile computing device. Connections between blood glucose meters and mobile computing devices are discussed elsewhere herein.

In many embodiments, a remote diabetes care provider and/or a diabetes care system may act on a user request based on blood glucose information and on other information. For instance, in many embodiments, the method can include receiving physiological and/or behavioral information from the user at the software application other than blood glucose information. In the method, the user can provide physiological and/or behavioral information to the software application (412). In some embodiments, the user may provide additional information (e.g., biographical information, insurance information, calendar information, etc.) to the software application. In some embodiments, the software application may pull various types of information from the mobile computing device.

In many embodiments, the method can verify that the blood glucose information is from the user. It may be important to verify that the blood glucose information is properly associated with the user so that the remote diabetes care provider and/or the diabetes care system is not acting on a user request based on information not properly associated with the user (e.g., someone other than the user provided the biological sample for the blood glucose measurement). The method can include providing bio-identification information. The user can provide bio-identification information to the software application (413). The blood glucose measurement can use a biological sample provided by the user. The bio-identification information can verify that the blood glucose information is from the user. In some embodiments, the bio-identification information can include a video of a blood glucose measurement using a biological sample provided by the user. In such embodiments, the video can show the user submitting his/her biological sample, along with the corresponding measurement. In some embodiments, DNA can be verified from the biological sample used in the blood glucose measurement.

In many embodiments, the method can involve prompting a user to make a request through the software application. For example, the diabetes care system may know that a user's prescription will soon expire or that the user is due for a driver's license renewal. The diabetes care system may prompt the user to make a request through the software application to renew the prescription or to grant medical clearance for the driver's license renewal. In some embodiments, the method can include receiving a prompt from the diabetes care system (420). The prompt can be received at the software application. In some embodiments, the method can include displaying the prompt on the mobile computing device. The prompt can be displayed using the software application.

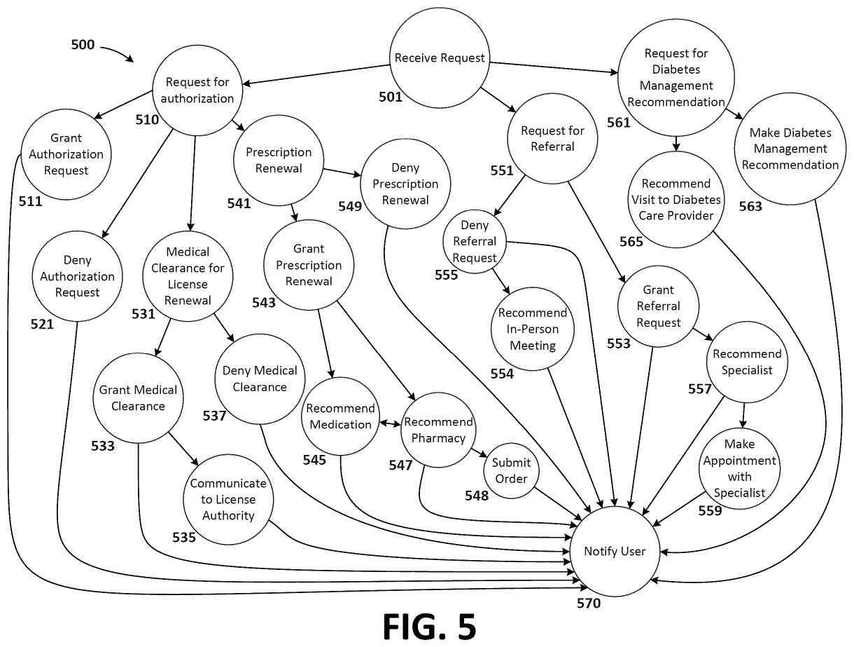

Whether prompted (431) or unprompted (441), the user can make a request. The user can make the request using the software application on the mobile computing device (432). Examples of requests made by users are discussed in various places herein, including in connection with FIG. 5.

When the software application receives the request, the software application can provide information and the request to the remote diabetes care provider through a diabetes care system (433). Such information can include blood glucose information, physiological and/or behavioral information, bio-identification information, and/or other relevant information stored in the software application.

The diabetes care system and/or the remote diabetes care provider can make a determination of whether the information provided is from the user and not from someone else (450). If there is inadequate confirmation that the information provided is from the user, the diabetes care system can respond by asking for additional bio-identification information (461). The software application can provide additional bio-identification information to the diabetes care system (462). These steps can repeat until the diabetes care system and/or the remote diabetes care provider are satisfied that the information provided by the software application is from the user and not from someone else.

When the diabetes care system and/or the remote diabetes care provider are satisfied that the information provided is from the user and not from someone else, a determination can be made whether the information is sufficient to respond to the request (470). If the information is not sufficient to respond to the request, the diabetes care system can request supplemental information (481). For example, the diabetes care system can request additional blood glucose information, physiological and/or behavioral information, and/or other relevant information. The software application can receive the supplemental information. The software application can provide the supplemental information to the diabetes care system (482). The remote diabetes care provider can access the supplemental information through the diabetes care system. In some embodiments, the supplemental information can include a photograph. The photograph can be of an anatomical part of the user (e.g., a photograph of the user's foot). In some embodiments, the supplemental information can include a video. The video can be of an anatomical part of the user. In some embodiments, the supplemental information can include results of a physiological test. The physiological test can be self-administered by the user (e.g., a self-administered vision test or tactile feedback test). These steps can repeat until the diabetes care system and/or the remote diabetes care provider have the information necessary to respond to the request.

In many embodiments, the diabetes care system can analyze the information received from the software application (485). As discussed elsewhere herein, the diabetes care system can run an algorithm on two or more information inputs and output a determination based on the inputs. The diabetes care system can provide analyzed information to the remote diabetes care provider. Information that can be analyzed by the diabetes care system can include the physiological and/or behavioral information and the blood glucose information.

When the diabetes care system and/or the remote diabetes care provider have the necessary information, the remote diabetes care provider can generate a response and provide the response to the diabetes care system (491). In some embodiments, the response can be based on the blood glucose information. When the initial response from the diabetes care system is a request for supplemental information, the remote diabetes care provider can generate a supplemental response based on the supplemental information. When the diabetes care system analyzes the information received from the software application and provides analyzed information to the remote diabetes care provider, the response can be based on the analyzed information.

The diabetes care system can send the response (and/or supplemental response) to the software application (492). In some embodiments, the response can include a notification. The software application can display the response on the mobile electronic device using the software application (493). Illustrative requests and responses are discussed in greater detail in connection with FIG. 5.

FIG. 5 shows a decision tree for receiving and responding to requests (500). FIG. 5 shows that a request may be received at the diabetes care system (501). In some embodiments, the request can include a request for an authorization from the diabetes care provider (510). The diabetes care provider can respond to the request by granting the authorization request (511) or denying the authorization request (521). In either case, the diabetes care system can notify the user of the diabetes care provider's response without the user having to meet with the diabetes care provider in person (570). As is discussed herein, in some instances, the diabetes care provider may request additional information from the user (e.g., blood glucose information, A1C information, blood pressure information, medication history, history of low blood sugar incidents, etc.) before acting on the request.

Requests for authorization can take a variety of forms. In many embodiments, the request for the authorization can include a request for medical clearance for purposes of renewing a license, for example (531). The diabetes care provider can respond to the request by granting the medical clearance (533) or denying the medical clearance (537). In either case, the diabetes care system can notify the user of the diabetes care provider's response without the user having to meet with the diabetes care provider in person (570). In some instances, the diabetes care system can not only grant the requested medical clearance (533) but also communicate the granted medical clearance to an authority responsible for renewing the license (535). The diabetes care system can notify the user that the granted medical clearance has been communicated to the license authority (570).

In many embodiments, the request for the authorization can include a prescription renewal request (541). The diabetes care provider can respond to the request by granting the prescription renewal (543) or denying the prescription renewal (549). In either case, the diabetes care system can notify the user of the diabetes care provider's response without the user having to meet with the diabetes care provider in person (570). In some instances, the diabetes care system can not only grant the prescription renewal request (543) but also recommend a medication for the user (e.g., based on medication price information (e.g., brand or generic), insurance information of the user, etc.) (545). In some instances, the diabetes care system can not only grant the prescription renewal request (543) but also recommend a pharmacy (e.g., based on pharmacy price information, location information of the user, etc.) (547). If the diabetes care system recommends a medication (545) and/or recommends a pharmacy (547), the diabetes care system can notify the user of such recommendations (570). In some embodiments, the diabetes care system can not only recommend a pharmacy (547) but also submit an order to the recommended pharmacy (e.g., of the recommended medication) (548). The diabetes care system can notify the user that the order was submitted to the recommended pharmacy (570).

In many embodiments, the request can include a request for a referral to a diabetes care specialist (551). The diabetes care provider can respond to the request by granting the referral request (553) or denying the referral request (555). In either case, the diabetes care system can notify the user of the diabetes care provider's response without the user having to meet with the diabetes care provider in person (570). In some instances, the diabetes care system can not only grant the referral request (553) but also recommend a specialist (e.g., based on physiological and/or behavioral information, location information, and insurance information, etc.) (557). If the diabetes care system recommends a specialist (557), the diabetes care system can notify the user with the information about the recommended specialist (570). In some such instances, the diabetes care system can not only recommend a specialist (557) but also make an appointment with the recommended specialist (e.g., based on calendar information) (559). If the diabetes care system makes an appointment with the recommended specialist (559), the diabetes care system can notify the user of the appointment (559).

As noted, in some instances, the diabetes care provider may deny the referral request (555). In many such instances, the diabetes care provider may recommend instead that the user meet with the diabetes care provider in person (554). Such a recommendation may be based on blood glucose information, physiological and/or behavioral information, location information, insurance information of the user, and/or other relevant factors. The diabetes care provider may decide to make a referral during the in-person meeting.

In some embodiments, the request can include a request for a diabetes management recommendation (561). For example, in the request, a user may indicate that he/she is not feeling well and may ask if there are steps he/she can take to feel better. Or users may ask if they can increase or decrease the amount of insulin they are taking. In some instances, the diabetes care provider can make a diabetes management recommendation (563). For example, the diabetes care provider may recommend that the user adjust his/her exercise level, adjust his/her insulin intake level, review specified educational materials, and/or take other steps to manage his/her diabetes (or pre-diabetes) more effectively. In some instances, the diabetes care provider may decline to make a diabetes management recommendation but may instead recommend that the user visit the diabetes care provider in person (565). Whether the diabetes care provider makes a diabetes management recommendation (563) or instead recommends an in-person visit to the diabetes care provider (565), the diabetes care system can notify the user (570).

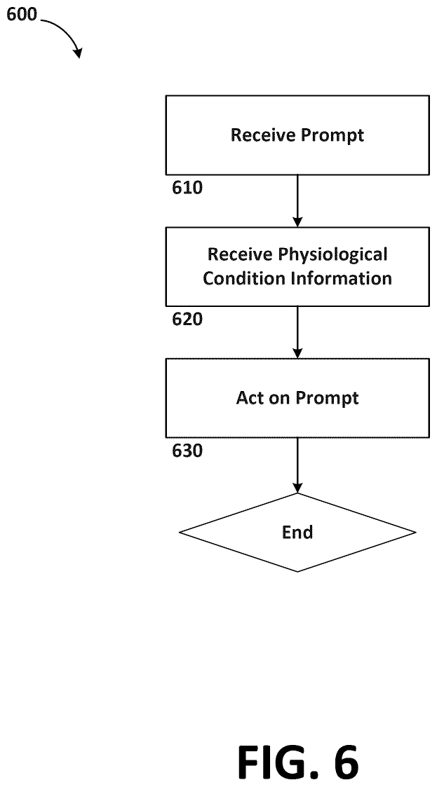

FIG. 6 illustrates a flow diagram of an exemplary embodiment of a process 600. The process 600 can be performed to act on a prompt for a healthcare provider authorization. For instance, the process 600 can allow for action to be taken in relation to the prompt for the healthcare provider authorization without requiring the patient to meet in-person with the healthcare provider at the healthcare provider's facility. In certain examples, the process 600 can be facilitated by an embodiment of the physiological condition information system disclosed herein.

At step 610, the process 600 includes receiving a prompt for a healthcare provider authorization concerning a particular patient. In one example, the prompt is generated by a component of a physiological condition information system, such as that described herein, and received at a healthcare provider. For instance, the prompt can be an electronic request for a healthcare provider authorization from a patient who is remote from the healthcare provider. The electronic request from the remote patient can be generated, for instance, at a physiological condition management device accompanying the remote patient and delivered to the healthcare provider. In another instance, the prompt is a reminder from the physiological condition information system that a remote patient is due for a healthcare provider authorization. As one such example, the prompt is an electronic request from a remote server that generates and sends the prompt to a healthcare provider according to a patient profile stored at the remote server. In this instance, the patient may set up the patient profile held at the remote server to automatically send the prompt to a healthcare provider for the healthcare provider authorization at an appropriate time according to the type of healthcare provider authorization that the patient needs. This can also include, additionally or alternatively, the remote server generating and sending the prompt to the remote patient reminding the patient that he or she is due for a healthcare provider authorization. In response, the remote patient may send the electronic request for a healthcare provider authorization to the healthcare provider.

The healthcare provider authorization at step 610 can take a variety of forms depending on the particular patient. As one example, the healthcare provider authorization can be an authorization from a medical professional that the patient meets the medical requirements for a license issuance or renewal. The license can be any type of license that is issued or renewed depending, at least in part, on the physiological condition of a person seeking the license. For example, when a person has a certain type of medical condition some licensing agencies require a medical professional to sign off that the person seeking the license has sufficient and stable control of this medical condition before a license can be issued or renewed. Such a medical condition can include, as examples, diabetes mellitus, dementia, seizure episodes, prior stroke, and arthritis. Examples of licenses that may require an authorization from a medical professional that the patient meets certain requirements for issuance or renewal can include, as examples, a driver's license, a pilot's license, an air traffic controller license, a nurse's license, and a teacher's license.

As another example, the healthcare provider authorization can be an authorization from a medical professional that a prescription be filled for the patient. The prescription can be any type of prescription that is filled for a patient only upon proof of healthcare provider authorization of the prescription fulfillment. This may include prescriptions for medications or devices.

As a further example, the healthcare provider authorization can be an authorization from a medical professional verifying that the patient has met specified medical conditions for employment or probation. For instance, a potential employer may require that a patient submit verification from a medical professional that certain substances are not present in the patient's system, such as is the case in employment drug tests. The same may be true for court ordered probation for a patient. In another example, the healthcare provider authorization can include a determination whether an individual should be receiving disability benefits.

At step 620, the process 600 includes receiving physiological condition information associated with the patient. In some cases, the physiological condition information can be received along with the prompt received at step 610. The physiological condition information may be generated by a component of a physiological condition information system, such as that described herein, and in some instances may also be received at a component of this physiological condition information system. The physiological condition information that is received can be any type of physiological condition information that may be relevant to rendering the healthcare provider authorization. Thus, the type of information that is received can vary depending on the patient and/or the type of healthcare provider authorization in step 610.

In one embodiment, at step 620 physiological condition information is received from a physiological condition management device, such as that described herein. The physiological condition management device can be configured to collect any physiological condition information associated with the patient. In some cases, multiple, different measurement devices can be included as part of the physiological condition management device. As such, the physiological condition management device can include any component useful in ascertaining a physiological condition of the patient that is relevant to the healthcare authorization in step 610. Such components that may be useful in collecting physiological condition information associated with the patient can include a camera, an activity measurement mechanism (e.g., an accelerometer), a location determination mechanism (e.g., GPS component), a heart rate monitor and/or a blood characteristic analytical device (e.g., a blood glucose management device).

In one application, the received physiological condition information associated with the patient includes blood glucose management information. In this application, the physiological condition management device includes a blood glucose management device or system. This blood glucose management device or system can be the same as or similar to those described in the publications referenced previously and incorporated herein by reference. In addition, the received blood glucose management information can have characteristics that are the same as or similar to those described in the publications referenced previously and incorporated herein by reference. For instance, the received blood glucose management information might include one or more blood sugar testing results, a number of blood sugar tests performed over a predetermined period of time (e.g., per day), glucose time in range, a number of glucose events, calculated A1C values, and patient-provided answers to prompted questions. Patient-provided answers may include information pertaining to the patient's understanding of diabetes management and/or information pertaining to how the patient is controlling his or her diabetes condition on a regular basis.

In another application, the received physiological condition information associated with the patient includes blood pressure information. In this application, the physiological condition management device includes (e.g., integral to the device or as a separate component in communication with the device) a blood pressure measurement device. For instance, the received blood pressure information can include one or more patient blood pressure measurements and a time at which each blood pressure measurement was taken. In some instances, a number of blood pressure measurements can be taken and recorded (e.g., at the device, at the remote server) so as to generate a historical log of the patient's blood pressure information over a predetermined period of time. In certain cases, this blood pressure information can be compared to a predetermined blood pressure range for the specific patient and if the blood pressure information falls outside of the range an alert can be generated and sent to the patient. In various examples, patient-provided answers can be included as the blood pressure information, for instance in addition to the patient blood pressure measurements. To obtain such answers, prompts can be presented to the patient relating to how the patient is controlling his or her hypertension on a regular basis and/or events relating to the patient occurring contemporaneous to acquired blood pressure measurements. Such events may relate to life activities of the patient, such as dietary events or subjectively stressful events. Having patient-provided answers to prompts may be useful in putting one or more objective blood pressure measurements in context. Moreover, such information can acquired without needing the patient present at the remote healthcare provider.

In other applications, the physiological condition information that is received can be any other type of physiological condition information that may be relevant to rendering the healthcare provider authorization. For instance, in other applications, the received physiological condition information associated with the patient can include information pertaining to a patient's dementia condition, seizure episodes, prior stroke related issues, or arthritis conditions. This information may include objective one or more physiological measurements and/or patient-provided answers to prompted questions.

At step 630, the process 600 includes acting on the prompt for a healthcare provider authorization concerning the patient. Acting on the prompt in step 630 can be performed without requiring the patient to meet with a medical profession rendering the healthcare provider authorization.

As one example, in step 630, acting on the prompt for the healthcare provider authorization includes granting the healthcare provider authorization. The healthcare provider authorization may be granted if the medical professional determines that the requested healthcare provider authorization is warranted by the received physiological condition information associated with the patient. For instance, this may include assessing whether received physiological condition information associated with the patient meets predetermined requirements relative to the patient and/or objectively set requirements. In one case, this may include assessing one or more trends in the received physiological condition information over one or more preceding periods of time.

As another example, in step 630, acting on the prompt for the healthcare provider authorization includes denying the healthcare provider authorization. The healthcare provider authorization may be denied if the medical professional determines that the requested healthcare provider authorization is not warranted by the received physiological condition information associated with the patient. For instance, this may include assessing whether received physiological condition information associated with the patient fails to meet predetermined requirements relative to the patient and/or objectively set requirements. In one case, this may include assessing one or more trends in the received physiological condition information over one or more preceding periods of time.

As a further example, in step 630, acting on the prompt for the healthcare provider authorization includes obtaining additional information about the patient. In one case, obtaining additional information about the patient can include asking the patient for additional information. In another case, obtaining additional information about the patient can include acquiring additional information from the physiological condition management device carried by the patient. This may include sending a data communication to the physiological condition management device carried by the patient and in response receiving a data communication from this physiological condition management device. In some cases, it may be possible to obtain needed additional information about the patient automatically from the physiological condition management device carried by the patient. An example of additional patient information that may be obtained includes an anatomical photograph of the patient acquired by the physiological condition management device carried by the patient. Such anatomical photographs may include, for instance, a photograph of the patient's teeth, a photograph of the patient's eyes, a photograph of the patient's foot, and/or a photograph of the patient's skin. Another example of additional patient information that may be obtained includes activity measurement and/or location determination associated with the patient.

In an additional example, at step 630, acting on the prompt for the healthcare provider authorization includes requesting the patient to meet with the healthcare provider. In one case, the request can be sent to the physiological condition management device carried by the patient. Step 630 can include requesting that the patient meet with a medical professional of a selected level of specialization. The level of specialization of the medical professional can be selected based on the received physiological condition information associated with the patient. For instance, the received physiological condition information associated with the patient can be used to determine that the patient should meet with a healthcare professional that is a specialist physician (e.g., an endocrinologist). In another instance, the received physiological condition information associated with the patient can be used to determine that the patient should meet with a healthcare professional that is a healthcare professional that is a generalist physician (e.g., a general practitioner). In a further instance, the received physiological condition information associated with the patient can be used to determine that the patient should meet with a healthcare professional that is less specialized than a physician (e.g., a physician assistant). In this way, healthcare costs may be reduced and resources of the healthcare provider can be efficiently allocated to meet the level of medical attention warranted by the received physiological condition information associated with the patient.

In another example, at step 630, acting on the prompt for the healthcare provider authorization includes communicating a decision on the prompt. Depending on the type of healthcare provider authorization requested by the prompt, the decision can be communicated to a variety of recipients. In one case, the decision on the prompt is communicated to the patient, such as by sending the decision to the physiological condition management device carried by the patient. In another case, the decision on the prompt is communicated to the remote server, such as to be stored in association with the corresponding patient profile at the remote server. In a further case, the decision on the prompt is communicated to a licensing body (e.g., a local governmental Department of Motor Vehicles or other governmental licensing body). In an additional case, the decision on the prompt is communicated to a prescription fulfillment site (e.g., a pharmacy).

In a further embodiment of the process 600, a step of receiving bio-identification information from the patient can be included. Bio-identification information can be received in addition to the physiological condition information associated with the patient 206. This may be useful in verifying that the collected physiological condition information is actually associated with the patient it is purported to be from. Such bio-identification information can include any biological information that can be used in authenticating a particular patient's identity. This may include, for example, finger print data, facial recognition data, implanted chip data signal reception, or any other suitable bio-identification information that can be provided by a patient. In one instance, bio-identification information can be received as part of the step 620.

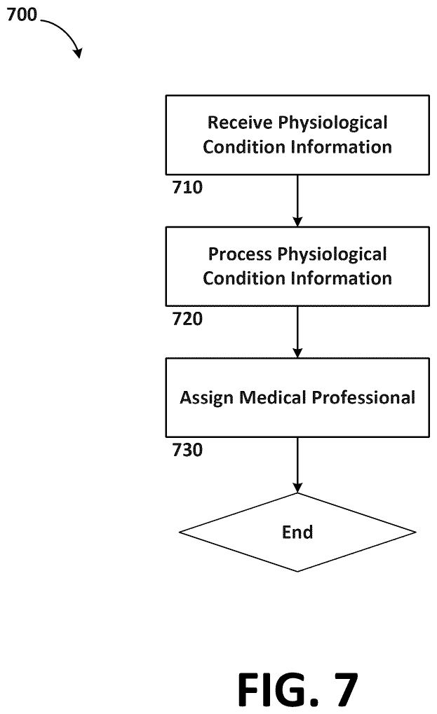

FIG. 7 illustrates a flow diagram of an exemplary embodiment of a process 700. The process 700 can be useful, for example, in automatically assigning a medical professional to a patient using physiological condition information associated with the patient.

At step 710, the process 700 includes receiving physiological condition information associated with a particular patient. This physiological condition information can include any physiological condition information associated with the patient, including the types of physiological condition information described elsewhere herein. The physiological condition information can be received from a patient who is located at a first location that is remote from a second location of a medical professional.

At step 720, the process 700 includes processing the received physiological condition information associated with the patient. This can include, for example, comparing the received physiological condition information associated with the patient to one or more predetermined thresholds relative to the patient and/or objectively set thresholds. In one case, this may include assessing one or more trends in the received physiological condition information over one or more preceding periods of time to determine an extent of any changes in the physiological condition information associated with the patient.

At step 730, the process 700 includes assigning a medical professional to the patient based on the processing of the received physiological condition information associated with the patient at step 720. In some cases, assigning a medical professional to the patient at step 730 can include selecting a level of specialization of the medical professional using the processed physiological condition information associated with the patient. In this way, medical provider resources can be used efficiently by appropriately pairing generally higher cost, more specialized medical professionals with those patients having physiological condition information indicating a need for this type medical attention. At the same time, generally, lower cost, less specialized medical professionals can be paired with those patients having physiological condition information indicating that the less specialized medical professional would be suitable to provide medical attention to the patient.

For example, a medical professional that is a specialized physician (e.g., an endocrinologist) may be assigned to meet with the patient where the processed physiological condition information associated with the patient indicates that the expertise of the specialized physician may be needed to treat the patient. A medical professional that is a generalist physician (e.g., a general practitioner) may be assigned to meet with the patient where the processed physiological condition information associated with the patient indicates that the expertise of the specialized physician may not be needed to treat the patient but that a medical professional more specialized than a non-physician may be needed. A medical professional that is less specialized than a physician (e.g., a physician assistant) may be assigned to meet with the patient where the processed physiological condition information associated with the patient indicates that the expertise of a non-physician can be suitable in treating the patient.

In one embodiment, step 730 may further include scheduling a patient appointment with the assigned medical professional. This may include processing available schedules of one or more medical professionals at the selected level of specialization and assigning a medical professional at the selected level of specialization based on available appointment time slots.

Any one or more of the techniques and processes described in this disclosure may be embodied or encoded in a non-transitory computer-readable medium, such as a computer-readable storage medium, containing instructions. Instructions embedded or encoded in a computer-readable storage medium may cause a programmable processor, or other processor, to perform the process, e.g., when the instructions are executed. Non-transitory computer readable storage media may include volatile and/or non-volatile memory forms including, e.g., random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described with reference to certain disclosed embodiments. The embodiments are presented for purposes of illustration and not limitation. One skilled in the art will appreciate that various changes, adaptations, and modifications can be made without departing from the scope of the invention.

What is claimed is:

1. A method of making a request of a remote diabetes care provider using a software application operating on a mobile computing device, the method comprising:
receiving blood glucose information from a user at the software application;
receiving the request at the software application;
providing the blood glucose information and the request from the software application to the remote diabetes care provider through a diabetes care system;
receiving a response to the request at the software application from the remote diabetes care provider through the diabetes care system without the user having to visit the remote diabetes care provider, the response being based on the blood glucose information; and
displaying the response on the mobile computing device using the software application.

2. The method of claim 1, wherein the software application receives the blood glucose information wirelessly from a blood glucose meter connected to the mobile computing device.

3. The method of claim 2, wherein the blood glucose information comprises blood glucose measurements collected from the user over a period of time.

4. The method of claim 1, wherein the request comprises a request for an authorization from the remote diabetes care provider.

5. The method of claim 4, wherein the request for the authorization comprises a request for medical clearance for purposes of renewing a license.

6. The method of claim 5, wherein the response comprises a notification that the request for medical clearance has been granted and communicated by the diabetes care system to an authority responsible for renewing the license.

7. The method of claim 4, wherein the request for the authorization comprises a prescription renewal request.

8. The method of claim 7, wherein the response comprises a recommended medication, wherein the diabetes care system identifies the recommended medication based on medication price information and insurance information of the user.

9. The method of claim 8, wherein the response comprises a notification that an order of the recommended medication was submitted to a recommended pharmacy, wherein the diabetes care system identifies the recommended pharmacy based on pharmacy price information and location information of the user.

10. The method of claim 4, wherein the response comprises a notification that the request for authorization has been granted or denied.

11. The method of claim 1, wherein the request comprises a request for a referral to a diabetes care specialist.

12. The method of claim 11, wherein the response comprises a grant of the request for the referral, along with a name of a recommended diabetes care specialist, wherein the diabetes care system identifies the recommended diabetes care specialist based on physiological and/or behavioral information, location information, and insurance information of the user.

13. The method of claim 12, wherein the response further comprises notification of an appointment for the user with the recommended diabetes care specialist, wherein the method further comprises receiving user calendar information at the software application and providing the user calendar information to the diabetes care system, wherein the diabetes care system communicates with the recommended diabetes care specialist and makes the appointment based on the user calendar information.

14. The method of claim 1, wherein the request comprises a request for a diabetes management recommendation.

15. The method of claim 14, wherein the response comprises a recommendation to visit a recommended diabetes care provider, wherein the diabetes care system makes the recommendation based on physiological and/or behavioral information, location information, and insurance information of the user.

16. The method of claim 1, wherein the response comprises a recommendation that the user visit the remote diabetes care provider.

17. The method of claim 1, wherein the response comprises a request for supplemental information about the user, the method further comprising:
receiving the supplemental information at the software application;
providing the supplemental information from the software application to the remote diabetes care provider through the diabetes care system;
receiving a supplemental response at the software application from the remote diabetes care provider through the diabetes care system without the user having to visit the remote diabetes care provider, the supplemental response being based on the supplemental information; and
displaying the supplemental response on the mobile computing device using the software application.

18. The method of claim 17, wherein the supplemental information comprises a photograph or video of an anatomical part of the user.

19. The method of claim 17, wherein the supplemental information comprises results of a physiological test self-administered by the user.

20. The method of claim 1, further comprising:
receiving a prompt from the diabetes care system at the software application; and
displaying the prompt on the mobile computing device using the software application, the request being responsive to the prompt.

21. The method of claim 1, further comprising:
providing bio-identification information from the software application to the remote diabetes care provider through the diabetes care system, the bio-identification information verifying that the blood glucose information is from the user.

22. The method of claim 21, wherein the bio-identification information comprises a video of a blood glucose measurement using a biological sample provided by the user.

23. The method of claim 1, further comprising:
receiving physiological and/or behavioral information from the user at the software application, the physiological and/or behavioral information being information about the user other than blood glucose information;
providing the physiological and/or behavioral information from the software application to the diabetes care system, wherein the diabetes care system analyzes the blood glucose information and the physiological and/or behavioral information and provides analyzed information to the remote diabetes care provider,
wherein the response is based on the analyzed information.
